(19) United States
(12) Patent Application Publication
(10) Pub. No.: US 2024/0390068 A1
(43) Pub. Date: Nov. 28, 2024

(54) SYSTEMS AND METHODS FOR GENERATING WORKSPACE GEOMETRY FOR AN INSTRUMENT

(71) Applicant: INTUITIVE SURGICAL OPERATIONS, INC., Sunnyvale, CA (US)

(72) Inventors: Steven Manuel, Sunnyvale, CA (US); Daniel H. Gomez, Los Gatos, CA (US); Daniel N. Miller, San Jose, CA (US); John Ryan Steger, Los Gatos, CA (US); Prasad V. Upadrasta, San Jose, CA (US); Zhuoqun Xu, Sunnyvale, CA (US); Chun hua Zheng, Santa Clara, CA (US); Pavel Chtcheprov, Apex, NC (US)

(73) Assignee: INTUITIVE SURGICAL OPERATIONS, INC., Sunnyvale, CA (US)

(21) Appl. No.: 18/670,002

(22) Filed: May 21, 2024

Related U.S. Application Data
(60) Provisional application No. 63/503,803, filed on May 23, 2023.

(51) Int. Cl. A61B 34/10; A61B 17/00; A61B 34/20; A61B 34/30; A61B 34/37; A61B 90/00

(52) U.S. Cl. CPC A61B 34/10; A61B 17/00234; A61B 34/20; A61B 34/25; A61B 34/37; A61B 90/361; A61B 2034/105; A61B 2034/107; A61B 2034/2048; A61B 2034/2051; A61B 2034/2055; A61B 2034/301; A61B 2034/302

(57) ABSTRACT

A computer-assisted system can comprise an instrument configured to be at least partially inserted through a body wall from an external workspace to an internal workspace within a body; and a controller configured to generate a first three-dimensional model of the internal workspace in which a first portion of the instrument is inserted during performance of a medical procedure using the instrument, generate a second three-dimensional model of an external workspace in which a second portion of the instrument is located during the performance of the medical procedure, based on the first three-dimensional model and the second three-dimensional model, determine an internal geometry within the internal workspace defining a reachable volume within the internal workspace within which the instrument may be positioned, and provide output related to performance of the medical procedure based on the determined internal geometry.

### CROSS-REFERENCE TO RELATED APPLICATIONS

This application claims priority to U.S. Provisional Application No. 63/503,803, filed May 23, 2023, the entire contents of which is incorporated herein by reference.

### FIELD

The present disclosure is directed to systems and methods for performing procedures using instruments without a direct line of sight of one or more workspaces in which the instrument or manipulator is deployed to perform the procedure. More particularly, aspects of the present disclosure relate to systems, methods, and devices for determining and/or generating workspace geometries for assistance in planning such procedures, such as for an instrument performing a minimally invasive medical procedure.

### INTRODUCTION

Benefits of minimally invasive medical procedures are well known, and they include less patient trauma, less blood loss, and faster recovery times when compared to traditional, open incision surgery. In minimally invasive medical procedures, one or more incisions may be made in a patient's body wall, with one or more instruments inserted through those incisions, either directly or via one or more other guide devices such as ports, cannulas, and the like. In some cases, all instruments used are passed through a single incision in the patient's body wall, or in some instances through a single natural orifice of a patient's body. In other cases, multiple incisions are made in the patient's body wall, multiple natural orifices are used, or combinations thereof, with differing instruments inserted in the different incisions/orifices. Moreover, minimally invasive medical procedures can be performed using instruments controlled at least in part through computer-assisted systems that employ robotic technologies (sometimes referred to as robotic systems and permutations thereof), via manually actuated instruments, or a combination of the two. In any of the above cases, the various instruments themselves and/or components of manipulating systems to which they are operably coupled in a computer-assisted system, have a potential to contact, collide or otherwise interfere one another, such as at the internal space in which the instruments are inserted through the body wall and/or externally due to the need to move the instruments and/or components of the manipulator system to adjust a positioning and orientation of the instruments internally during a medical procedure. Such potential for interference limits the amount of instrument internal reach available to the surgeon. Moreover, it may be difficult for a surgeon (or other personnel controlling the instruments to perform the medical procedure) to know if one or more of the instruments and/or multiple manipulators in the case of a computer-assisted procedure are at risk of making contact with (such as colliding with or other undesirable contact) the patient or other obstacles in the environment surrounding the patient.

In addition, depending on the medical procedure being performed and on patient body type, there is variability in the placement of the one or more incisions and/or natural orifices to insert the one or more instruments, and thus the one or more ports placed at those incisions. If such placement of where on the patient's body an instrument is to be inserted is not properly planned, the medical procedure may be impacted. For example, placing two ports in a particular location relative to one another may increase the likelihood of undesirable contact of the instruments inserted at those locations (between one another, between the instruments and the patient, and/or between the instruments and external objects) and/or may decrease the reachable volume of the instrument within the internal workspace where the procedure of interest is occurring. In the case of a medical procedure performed using a robotic medical system having a plurality of manipulator arms connected to and providing drive input to control movement and other functionality of the instruments, the manipulator arms could be prone to contact (e.g., collision) in a similar manner. Undesired or unintended contact can lengthen the time of the medical procedure and/or result in having to redo incision/port placement and reinsertion of instruments.

A need exists for improvements relating to the planning and carrying out of medical procedures to address one or more of the issues noted above, and others that will be appreciated from the following description.

### SUMMARY

Various aspects of the present disclosure may solve one or more of the above-mentioned problems and/or may demonstrate one or more of the above-mentioned desirable features. Other features and/or advantages may become apparent from the description that follows.

In one aspect of the present disclosure, there is provided a computer-assisted system, comprising: an instrument configured to be at least partially inserted through a body wall from an external workspace to an internal workspace within a body; and a controller configured to: generate a first three-dimensional model of the internal workspace in which a first portion of the instrument is inserted during performance of a medical procedure using the instrument, generate a second three-dimensional model of an external workspace in which a second portion of the instrument is located during the performance of the medical procedure, based on the first three-dimensional model and the second three-dimensional model, determine an internal geometry within the internal workspace defining a reachable volume within the internal workspace within which the instrument may be positioned, and provide output related to performance of the medical procedure based on the determined internal geometry.

In another aspect of the present disclosure, there is provided a computer-implemented method, comprising: generating a first three-dimensional model of an internal workspace of a body in which a first portion of an instrument operatively coupled to a manipulator arm configured to transmit drive force to the instrument is inserted during performance of a procedure using the instrument; generating a second three-dimensional model of an external workspace in which a second portion of the instrument is located during the performance of the procedure; based on the first three-dimensional model and the second three-dimensional model, determining an internal geometry within the internal workspace defining a reachable volume within the internal workspace within which the instrument may be positioned by the manipulator arm, and providing output related to performance of the procedure based on the determined internal geometry.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more aspects of the present teachings and together with the description explain certain principles and operation. In the drawings:

FIG. 1 illustrates a view of an environment in accordance with various embodiments;

FIGS. 2A-2B respectively illustrate views of components of a robotic surgical system in accordance with various embodiments;

FIG. 3 illustrates a block diagram of an exemplary surgical system in accordance with various embodiments;

FIG. 4 illustrates an exemplary process flow in accordance with various embodiments;

FIGS. 5A-5B respectively illustrate views of an exemplary surgical system configuration in accordance with various embodiments;

FIG. 6 illustrates another exemplary process flow in accordance with various embodiments;

FIG. 7 illustrates an exemplary image view in accordance with various embodiments;

FIG. 8 illustrates yet another exemplary process flow in accordance with various embodiments; and

FIG. 9 illustrates an exemplary user interface display screen in accordance with various embodiments.

### DETAILED DESCRIPTION

As noted above, in minimally invasive medical procedures, whether performed manually (e.g., laparoscopically), via a computer-assisted surgical system utilizing robotic technology, or a combination of the two, the use of instruments inserted through one or more ports may lead to the risk of undesirable contact, such as, but not limited to, between multiple instruments, between manipulator arms coupled to such instruments, and/or between an instrument and another object external to the body in which the instrument is inserted (including, e.g., a patient or personnel). It may be difficult for medical personnel, such as a surgeon or other assisting personnel, to know if a risk of undesirable contact as noted above is occurring or about to occur. Even in cases in which feedback, such as haptic feedback or other feedback, is provided to notify of an undesirable contact, such feedback is generally provided via a channel that also conveys other information unrelated to such contact, which may render the feedback ambiguous. Moreover, particularly in the case of a computer-assisted surgical system, external contact with the body, personnel, or other objects that may provide a softer contact, may not be sufficiently perceptible to the personnel controlling the manipulation of one or more instruments to perform the medical procedure. Further, reduced visibility due to drapes, external objects impairing line of sight, body curvature, and the like, can make it difficult to observe, such as via assisting personnel, the occurrence or potential occurrence of contact between various objects external to the patient's body.

Various embodiments herein thus provide the ability for a surgeon to understand the location of boundaries within an internal workspace which would result in an external collision or other defined contact that it may be desirable to avoid if the surgeon were to attempt to reach an area beyond the boundaries.

Furthermore, where multiple ports are used, there is variability in how the ports are placed. In some situations, the port placement may impact the medical procedure itself, for example by resulting in contact of the arms and/or an inability to reach certain portions of the medical workspace. Thus, there exists a need for systems, devices, and methods for determining internal reach geometries which would result in an external contact and/or for determining and generating port placement guidance output.

Various embodiments herein thus also provide the ability for a surgeon to place ports in a configuration that may increase a size of a reachable volume within the internal workspace while reducing the likelihood of an external contact that may not be desired. The ports may be planned and placed in an iterative manner to ensure the reachable volume is sufficient for the procedure while using a minimum number of ports. While the following description is presented with reference to a single-cart (manipulator system) multi-port system, this is for purposes of explanation only and not limitation. The methods, operations, and implementations described herein may also be applied with a single-cart single-port system, a multi-cart multi-port system, a table-mounted single-port system, a table-mounted multi-port system, a wall-mounted single-port system, a wall-mounted multi-port system, a ceiling-mounted single-port system, a ceiling-mounted multi-port system, and the like.

Various embodiments further may provide the ability to update the internal reach geometry based on conditions changing during the medical procedure, such that the external workspace and internal workspace are dynamically updated and mapped to provide updated internal reach geometries throughout the medical procedure.

The terms “internal” and “external” are, in this context, used relative to a body such that the external workspace may at least partially surround the body and internal workspace and may correspond to the ambient environment from which an instrument is inserted into the internal workspace and controlled. In the context of a medical procedure, the boundary between the internal and external workspaces may correspond to or comprise an external surface portion of a patient body, such as a body wall through which an instrument is inserted, for example through a port or a natural orifice of the body. In non-medical contexts, a boundary between internal and external workspaces may correspond to a body of an object, such as but not limited to an opening of a pipe, the opening of a well, etc. through which an instrument is inserted to perform a nonmedical procedure, such as inspection, maintenance etc., and be used under the same principles of operation as described herein in the medical procedure context. As used herein, “medical procedure” may refer to a diagnostic procedure, a surgical procedure, a therapeutic procedure, and the like.

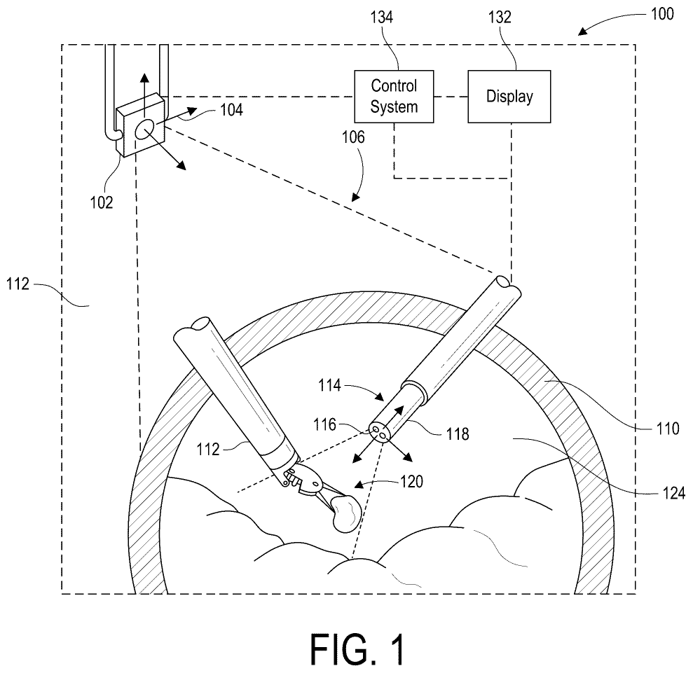

With reference now to FIG. 1, a schematic illustration of a system deployed within a medical procedure environment 100 for performing a minimally invasive medical procedure is shown and will be described. In FIG. 1, the system includes an external image sensing system comprising one or more external image sensors 102 (which may be mounted to, e.g., a manipulator assembly, a ceiling, a wall, or any of one or more of a variety of locations in an external environment in which a patient is located for performing the medical procedure) positioned to provide an image of an external workspace 122 and an internal image sensing system (e.g., comprising one or more endoscope instruments 114) positioned to provide an image of an internal workspace 124. The external image sensor 102 is associated with an external image sensor reference frame 104 and has a field of view 106. The external image sensing system comprising one or more external image sensors 102 may provide an external image of at least a portion of a patient body through which at least a portion of a medical instrument 112 and the endoscope instrument 114 are inserted to perform the medical procedure in the internal workspace 124, with the uninserted portions of the instruments 112, 114 (or cannulas through which they are inserted) being visible in the external workspace image. The instrument 112 includes an end effector that is controllable to perform a desired function of the instrument 112 (e.g., cutting, grasping, suturing, sensing, applying electrical energy, etc.) and a wrist that is operable to control a movement and/or orientation of the end effector. In an embodiment where the external image sensor 102 is located on an arm of a manipulator assembly, the external image provides an egocentric view from the manipulator assembly, and may provide an effective view for tracking various external objects including external portions of the medical instrument 112 and/or the endoscopic instrument 114. As shown in FIG. 1, the internal image sensor 116 provided at the distal end of endoscope instrument 114 is positioned to provide an image of the internal workspace 124 using an internal image sensor reference frame 118 and has a field of view 120. The internal image sensor 116 may provide an internal image of the interior of the patient body surrounded at least in part by the body wall 110 of the patient through which the instruments 112, 114 are inserted to perform a medical procedure at a worksite.

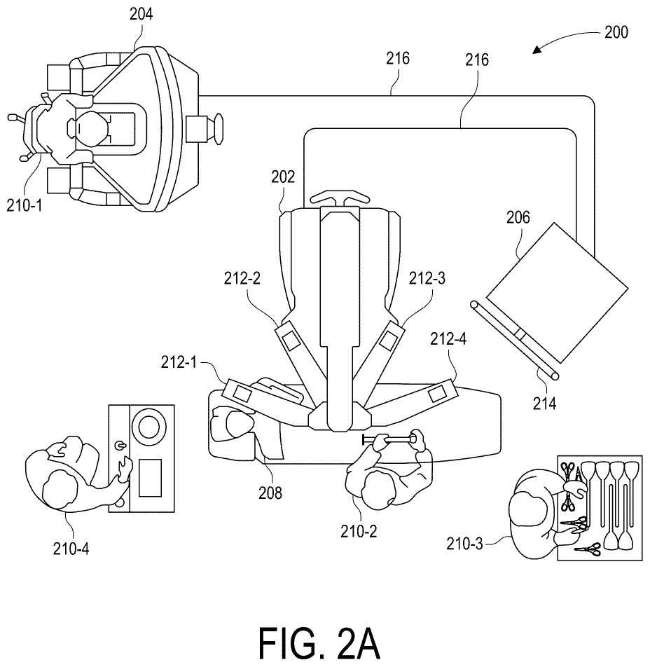

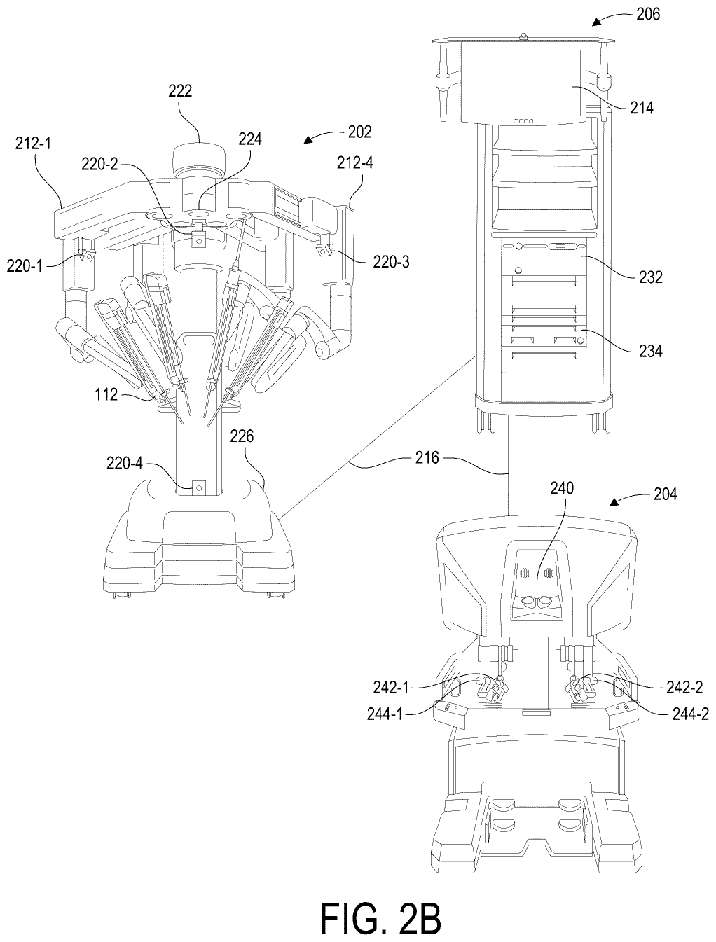

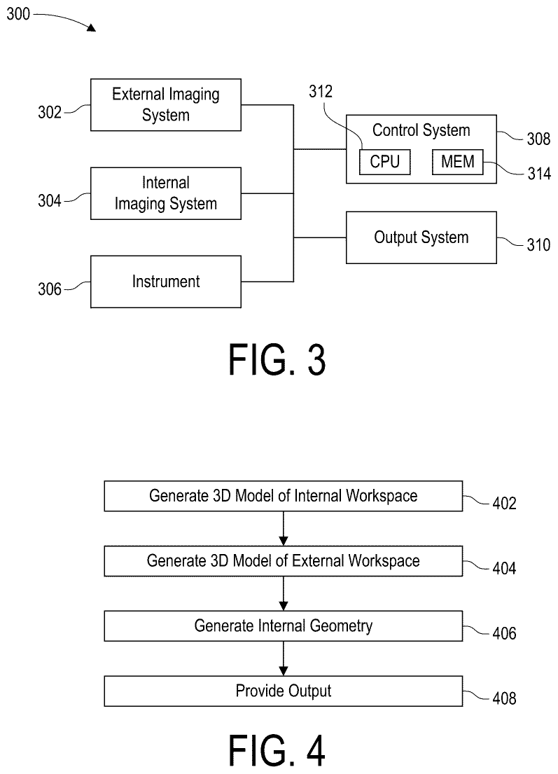

As described above, one or more medical instruments 112 are inserted through an incision in a body wall 110 of a patient to access the internal workspace 124 in which it is desired to perform one or more medical procedures. In some procedures, the instrument 112 is inserted through a guide device, such as a cannula, port body, trocar, or combination thereof, that are first inserted into the incision and provide passageway to insert the instrument from the external workspace 122 at least partially surrounding the body wall 110 to the internal workspace 124. To provide visualization of the internal workspace 124 to medical personnel performing the procedure, the internal image sensor 116 (which may be a part of an instrument such as an endoscope 114) also is inserted through the same or another incision in the body wall 110 into the internal workspace 124 and relays images of the internal workspace 124 to a display 132 (see also FIGS. 2A and 2B, described in more detail below) visible by medical personnel and operatively coupled to the endoscope 114 so as to receive internal image data. Throughout the disclosure, the term “port” and variations thereof is used to broadly describe a location on the body wall 110 or a natural orifice of a patient body through which an instrument 112/114 may be inserted, which can include directly through an incision or natural orifice, or through a guide device inserted into the incision or natural orifice, with the instrument inserted through the guide device. A control system 134 (which may be configured as described in more detail below with reference to FIG. 3) comprising a processor also is provided and in communication with the imaging sensing systems including image sensors 102, 116 so as to receive image data transmitted by those systems.

The system of FIG. 1 may be implemented for use in manual medical systems, computer-assisted (robotic) medical systems, or combinations of both types of systems. For example, in manual medical systems, one or both of the medical instrument 112 and the endoscopic instrument 114 (as well as one or more additional instruments not shown in FIG. 1) may be operated manually, such as by manual input from an operator (e.g., a surgeon or other medical personnel) at a force transmission mechanism provided with various inputs at a backend (proximal end portion not shown in FIG. 1) of the instrument 112, 114. In a computer-assisted medical system that relies in part on robotic technologies, one or both of the medical instrument 112 and the endoscopic instrument 114 (as well as one or more additional instruments not shown in FIG. 1) may be operated via the use of robotic technologies in which drive input forces (i.e., actuation forces) are provided at a force transmission mechanism provided with various inputs at a backend (proximal end portion not shown in FIG. 1) of the instrument 112, 114 configured to operably couple to and receive drive force from outputs of a manipulator interface of a manipulating system.

One example of a computer-assisted, robotic medical system and implementation is shown with reference to the schematic illustrations of FIGS. 2A and 2B.

As shown in FIGS. 2A and 2B, a computer-assisted, robotic medical system 200 in accordance with various embodiments, may include a manipulating system 202, a user control system 204, and an auxiliary system 206 communicatively coupled to one another. The medical system 200 may be utilized by a medical team to perform a computer-assisted medical procedure on a patient 208 (shown in FIG. 2A). As shown, the medical team may include one or more of a surgeon 210-1, an assistant 210-2, a nurse 210-3, and an anesthesiologist 210-4, all of whom may be collectively referred to as “medical team members 210.” Additional or alternative medical team members may be present during a medical procedure.

As used herein, a medical procedure can include any of a surgical procedure, a diagnostic procedure, a therapeutic procedure, a sensing procedure, and the like, and the medical procedure not only includes an operative phase of the procedure, but may also include preoperative (which may include setup of the medical system), postoperative, and/or other suitable phases of the medical procedure. In some implementations, one or more of the preoperative, operative, postoperative, and/or other phases of the medical procedure may include one or more stages. For example, different stages of the operative phase of a medical procedure may be associated with the use of different instruments.

As shown in FIG. 2A, manipulating system 202 may include a plurality of manipulator arms 212 (e.g., manipulator arms 212-1 through 212-4) to which a plurality of medical instruments 112 may be operably coupled. Each instrument 112 may be configured with a particular functionality (e.g., a tool having tissue-interaction functions such as but not limited to cutting, suturing, ablating, sealing, etc.), imaging device (e.g., an endoscope, an ultrasound tool, etc.), sensing instrument (e.g., a force sensing, pressure sensing, etc.), diagnostic instrument, or the like, that may be used for a computer-assisted medical procedure on patient 208 (e.g., by being at least partially inserted into patient 208 and manipulated by the manipulating system 202 to perform a computer-assisted medical procedure on patient 208). While manipulating system 202 is depicted and described herein as including four manipulator arms 212, it will be recognized that manipulating system 202 may include only a single manipulator arm 212 or any other number of manipulator arms as may serve a particular implementation. Moreover, in some implementations a combination of instruments 112 operated by manipulator arms 212 and instruments operated manually may be utilized. For example, FIG. 2A shows the patient being operated on by four medical instruments 112 operatively connected to the four manipulator arms 212-1 to 212-4 while the assistant 210-2 operates an endoscope manually. In other examples, the endoscope may be operated by one of the manipulator arms 212 while the assistant 210-2 (or multiple assistants) operate one or more medical instruments. In still other examples, all medical instruments and the endoscope may be manually operated, as mentioned above.

Manipulator arms 212 and/or instruments attached to manipulator arms 212 may include one or more displacement transducers, orientation sensors, and/or position sensors used to generate raw (i.e., uncorrected) kinematics information. One or more components of the system 200 may be configured to use the kinematics information to track (e.g., determine poses of) and/or control the instruments, as well as anything connected to the instruments and/or manipulator arms. The manipulator arms 212 may include a kinematic series of links connected by joints, with an instrument coupled to a distal link in the kinematic series. Individual joints and kinematic linkages may be powered or unpowered, and collectively may provide a plurality of different motions to orient the instrument in the internal workspace, with portions of the kinematic structure of the manipulator and a proximal portion of the instrument, including a force transmission mechanism operably coupled to receive drive input from the interface of the manipulator arm, moving in the external workspace. While FIGS. 2A and 2B show the manipulator arms 212 being attached to a central post 222 of the manipulating system 202, in other implementations the manipulator arms 212 may be attached to the ceiling or wall of the operating room, to the operating table which also supports the patient 208, to another mounting element, or other suitable attachment locations.

In the detailed view shown in FIG. 2B, the manipulating system 202 is shown with external image sensors 220 (image sensors 220-1 through 220-4) attached to components of the manipulating system 202. The external image sensors 220 may be used in addition to or as an alternative to the external image sensor 102 of FIG. 1, and may have the same or a similar structure. As shown, image sensor 220-1 may be attached to an orienting platform 224 of manipulating system 202, image sensor 220-2 may be attached to manipulating arm 212-1 of manipulating system 202, image sensor 220-3 may be attached to manipulating arm 212-4 of manipulating system 202, and image sensor 220-4 may be attached to a base 226 of manipulating system 202. In implementations in which manipulating system 202 is positioned proximate to a patient (e.g., as a patient side cart), placement of image sensors 220 at strategic locations on manipulating system 202 provides varied imaging viewpoints proximate to a patient and of a medical procedure performed on the patient.

In implementations (e.g., using a single-cart multi-port system as in FIG. 2B or one of the other system architectures noted above), one or more external image sensors 220 may be mounted at other locations, such as mounted to stands, walls, ceilings, and the like. In implementations, one or more external image sensors 220 may be a removeable device capable of temporary attachment (e.g., to the cart, to other locations, or to the manipulator arms 212). In other implementations, one or more external image sensors 220 may be handheld. In such implementations, the external image sensors 220 may be installed and/or operated only when a user desired to scan or re-scan the patient, the external workspace 122, or both. Moreover, in a single port system, implementations may have somewhat reduced complexity in terms of the boundaries to consider and display and the overall ranges of motions of instruments and/or a manipulator in the external workspace.

User control system 204 may be configured to facilitate control by surgeon 210-1 of manipulator arms 212 and the medical instruments 112 attached to manipulator arms 212. For example, surgeon 210-1 may interact with user control system 204 to remotely move or manipulate manipulator arms 212 and thus the instruments 112 operably coupled to the drive outputs of the manipulator arms 212. User control system 204 also may provide surgeon 210-1 with images (e.g., high-definition 3D images) of the internal workspace (e.g., internal workspace 124 of FIG. 1) associated with patient 208 as captured by an internal imaging system (e.g., an endoscope 114 which may be mounted to one of the manipulator arms or held manually). In certain examples, user control system 204 may include a stereo display (240 in FIG. 2B) having two displays where stereoscopic images of a surgical site associated with patient 208 and generated by a stereoscopic imaging system may be viewed by surgeon 210-1. Surgeon 210-1 may utilize the images displayed by user control system 204 to perform one or more procedures with one or more medical instruments 112 attached to manipulator arms 212.

To facilitate control of mounted instruments 112, user control system 204 may include a set of input controls 242 (e.g., a left input control 242-1 and a right input control 242-2) as can be seen in FIG. 2B. These input controls 242 may be manipulated by surgeon 210-1 to control movement of the instruments 112 (e.g., by utilizing robotic and/or teleoperation technology). The input controls 242 may be configured to detect a wide variety of hand, wrist, and finger movements by surgeon 210-1. In this manner, surgeon 210-1 may intuitively perform a procedure using one or more surgical instruments. The user control system 204 also includes feedback mechanisms 244 (e.g., a left feedback mechanism 244-1 and a right feedback mechanism 244-2), which may be equipped to provide haptic feedback to the surgeon 210-1.

Auxiliary system 206 may include one or more computing devices configured to perform processing operations of the medical system 200. In such configurations, the one or more computing devices included in auxiliary system 206 may control and/or coordinate operations performed by various other components (e.g., manipulating system 202 and user control system 204) of the surgical system. For example, a computing device included in user control system 204 may transmit instructions to manipulating system 202, based on the inputs at the input controls 242 of the user control system 204, by way of the one or more computing devices included in auxiliary system 206. As another example, auxiliary system 206 may receive and process image data representative of images captured by one or more external and/or internal image sensors.

In some examples, auxiliary system 206 may be configured to present visual content, such as for access by medical team members 210 who may not have access to the images provided to surgeon 210-1 at the display 240 of user control system 204. To this end, auxiliary system 206 may include a display monitor 214 configured to display one or more user interfaces, such as images of the internal workspace, information associated with patient 208 and/or the surgical procedure, and/or any other visual content as may serve a particular implementation. For example, display monitor 214 may display images of the internal workspace together with additional content (e.g., graphical content, contextual information, etc.) concurrently displayed with the images. In some embodiments, display monitor 214 is implemented by a touchscreen display with which team members 210 may interact (e.g., by way of touch gestures) to provide user input to the medical system 200.

As shown in FIG. 2B, the auxiliary system 206 also includes a central electronic data processing unit 232 and vision equipment 234. The central electronic data processing unit 232 includes circuits, devices, and/or other components to implement some or all of the data processing operations used to operate the surgical system, including but not limited to those operations described in more detail below. In other implementations, however, at least a portion of the data processing operations may be distributed in the user control system 204 and/or the manipulating system 202. The vision equipment 234 may include camera control units for the left and right image capture functions of the stereoscopic endoscope 114 (see FIG. 1). The vision equipment 234 may also include illumination equipment (e.g., a Xenon lamp) that provides illumination for imaging the internal workspace and/or external image capture equipment (e.g., image sensors similar to or the same as the image sensors 220) that captures an image of an external workspace within which the surgical site is situated. As illustrated in FIG. 2B, the auxiliary system 206 includes the monitor 214, however, in practical implementations the monitor 214 may be mounted elsewhere, such as on the manipulator system 202 or on a wall or ceiling of the operating room.

Manipulating system 202, user control system 204, and auxiliary system 206 may be communicatively coupled to one to another in any suitable manner. For example, as shown in FIGS. 2A and 2B, manipulating system 202, user control system 204, and auxiliary system 206 may be communicatively coupled by way of control lines 216, which may represent any wired or wireless communication link as may serve a particular implementation. To this end, manipulating system 202, user control system 204, and auxiliary system 206 may each include one or more wired or wireless communication interfaces, such as one or more local area network interfaces, Wi-Fi network interfaces, cellular interfaces, etc.

In certain examples, imaging devices such as external image sensor 102 may be attached to other components of the surgical system and/or components of a surgical facility where surgical system is set up. For example, image sensors 220 may be attached to components other than manipulating system 202. Accordingly, kinematics information for components of manipulating system 202 may be used by the system to derive kinematics information for the attached image sensors after a one-time calibration has been performed to identify relationships between tracked kinematics of components of manipulating system 202 and image sensors attached to the components of manipulating system 202. While the above description refers to an example in which the internal image sensor 116 is a stereoscopic image sensor and is part of an endoscope 114, in embodiments the internal image sensor 116 may take any form capable of determining a three-dimensional image. For example, the internal image sensor 116 may be, without limitation, a stereoscopic image sensor, a parallax image sensor, a time-of-flight (TOF) sensor, a light detection and ranging (LiDAR) sensor, a structured light camera, and the like. Additionally or alternatively, in some implementations the internal image may be obtained using external scanning or imaging equipment, including but not limited to a 3D x-ray imaging systems. Moreover, while the above description shows the external image sensors 102 and 220 as being single sensors for capturing a two-dimensional image, in embodiments the external image sensors 102 and/or 220 may take any form or combination of forms capable of collectively determining a three-dimensional image. For example, any of the external image sensors 102/220 may be, without limitation, one or more individual complementary metal-oxide semiconductor (CMOS) sensors, charge-coupled devices (CCDs), stereoscopic image sensors, parallax image sensors, TOF sensors, LiDAR sensors, structured light sensors, infrared (IR) sensors, sonar sensors, radar sensors, touch probes (e.g., touch sensors mounted on a manipulator arm), and the like.

The medical system 200 also may include components configured to sense and/or detect various signals. For example, the medical system 200 may include one or more position or orientation sensors to determine the location, orientation, speed, acceleration, etc. of various components of the medical system 200, such as the arms 212, the instruments, and/or the patient. The position or orientation sensors may include accelerometers, gravitometers, gyroscopes, Hall sensors, magnetometers, and the like. In some implementations position or orientation sensing may be performed using the image sensors 102/220, for example by detecting an insignia (e.g., a QR code) on an instrument, which may be helpful to assist with registering the external workspace with the internal workspace through the vectorization of the instrument for use in various implementations in accordance with the present disclosure as described below. In embodiments, the medical system 200 may perform registration between the external workspace and the internal workspace using the systems and methods discussed in International Patent Application No. PCT/US2021/065444, filed on Dec. 29, 2021, now published as WO 2022/147074, the entire contents of which are herein incorporated by reference.

FIG. 3 illustrates a block diagram of an embodiment of a medical system comprising the components of FIG. 1 and, in the case of a computer-assisted medical system employing robotic technologies, the components of FIGS. 2A and 2B. The system shown in FIG. 3 may be used to perform various aspects of the medical procedure relating to utilizing three-dimensional representations and registering of the internal and external workspaces, such as, but not limited to, port placement planning, defining internal workspace reach volumes, control of instrument and/or manipulator system movement to avoid contact between objects, providing feedback to medical personnel to assist with one or more of the above, or various combinations thereof. As illustrated, the system 300 comprises an external imaging system 302 (which may, for example, correspond to the external image sensor 102 and/or image sensors 220), an internal imaging system 304 (which may, for example, correspond to internal image sensor 116), at least one instrument 306, a control system 308, and an output system 310. While FIG. 3 illustrates the various systems and devices as being separate components, this is merely for purposes of illustration and in practical implementations, various components may be integrated with one another. For example, the control system 308 and the output system 310 may be components of the same device.

The control system 308 is illustrated as including a processor 312 and a memory 314. The processor 312 may include components (e.g., circuits and circuitry) configured to control other elements of the medical system, to process and/or execute instructions received from the memory 314 or other sources, to perform various method operations (including but not limited to those described herein), to apply algorithms to analyze data, to perform calculations and/or predictions, and the like. In some examples, the processor 312 may be or include one or more electronic processing units, such as central processing units (CPUs), arithmetic logic units (ALUs), floating-point units (FPUs), and/or other microcontrollers. The memory 314 may include components (e.g., circuits and circuitry) configured to store and/or receive information, including but not limited to computer readable instructions that, when executed by the processor 312, cause the control system 308 to perform various operations. The memory 314 may be or include one or more storage elements such as Random Access Memory (RAM), Read-Only Memory (ROM), optical storage drives and/or disks, magnetic storage drives and/or tapes, hard disks, flash memory, removable storage media, and the like. The control system 308 may include additional components, such as communication circuitry configured to allow communication (i.e., transmission and reception) with other components and devices. The communication circuitry may provide physical and/or virtual interfaces and communication ports for performing wired communication, wireless communications via radio transmission, optical communication via fiber or free electromagnetic radiation, and the like. The communication circuitry may further provide for connections to peripheral devices, such as Universal Serial Bus (USB) devices. In embodiments, the output system 310 may be an input/output (I/O) system capable of receiving input from a user. Thus, the output system 310 may include components configured to allow interaction with a user, including but not limited to the presentation of information to the user (e.g., via a display such as the display 132 of FIG. 1 and/or the monitor 214 of FIGS. 2A-2B to provide visual feedback, speakers to provide audio feedback, haptic feedback devices to provide haptic feedback, and the like) and/or the receipt of information from the user (e.g., via a touch screen, microphone, camera or other gesture detection device, physical buttons, soft buttons, and the like). The output system 310 may include a user interface, which may include or generate one or more graphical user interfaces (GUIs) and associated elements such as icons, menus, images, and the like. The user interface may be configured to report the results of calculations and/or predictions generated by the controller to the user.

The processor 312 is programmed with instructions to generate various 3D models (e.g., three-dimensional models of the internal workspace 124 and the external workspace 122), to determine various geometries based thereon, and to provide output (e.g., via the output system 310). The processor 312 may also be configured to control and/or plan various aspects of medical procedures based on models and geometries as will be discussed further with regard to various embodiments below.

In one example corresponding to the environment 100 of FIG. 1, the processor 312 may be configured to generate a first three-dimensional model of an internal workspace 124 in which a first portion of an instrument 112 is inserted during the performance of a medical procedure using the instrument 112, and to generate a second three-dimensional model of an external workspace 122 in which a second portion of the instrument 112 is located during the performance of the medical procedure using the instrument 112. The processor 312 may further be configured to, based on the first three-dimensional model and the second three-dimensional model, determine an internal geometry within the internal workspace defining a reachable volume within the internal workspace within which the instrument 112 may be positioned and to provide output related to performance of the medical procedure based on the determined internal geometry.

While various embodiments contemplate the systems and related components of FIGS. 1 and 2A-2B, and related methodologies carried out by those systems and as further described below can be implemented in the context of manually actuated instruments (e.g., such as, for example, in laparoscopic or endoscopic medical procedures using hand-held and manipulatable instruments), various embodiments herein contemplate medical procedures and the one or more medical instruments, imaging systems, and other components of FIGS. 1 and 2A-2B, are implemented as part of a computer-assisted medical system employing robotic technologies.

FIG. 4 illustrates an exemplary process flow which may be used, for example, to generate a reachable workspace geometry. The reachable workspace geometry may refer to the overall volume of the internal workspace which may be reached by the one or more inserted instruments. In various embodiments, the reachable workspace geometry may be determined through the application of differing criteria, such as, but not limited to, one or more of the physical constraints on an inserted instrument's ability to reach a given location within the internal workspace from its insertion through the external workspace and port location of the body wall, prevention of contact of one or more inserted instruments and/or manipulator arms with each other or other objects in the external workspace (e.g., patient body, medical personnel, other medical equipment, sterile drapes, and/or other environmental objects), and prevention of contact of one or more inserted instruments with objects in the internal workspace (e.g., another inserted instrument, an implant, an internal organ, a bony structure, and/or a cartilaginous structure). The process flow of FIG. 4 may be performed by, for example, the control system 308. In some implementations, the process flow of FIG. 4 may be embodied in the form of instructions stored in a non-transitory computer-readable medium (e.g., the memory 314) such that the control system 308 performs the operations of FIG. 4 upon execution of the instructions. In other implementations, the process flow of FIG. 4 may be embodied in the form of carrier signals (e.g., provided to the processor 312) such that the control system 308 performs the operations of FIG. 4 upon receipt of the instructions. For purposes of explanation, the operations of FIG. 4 will be described as being performed in/on the environment 100 of FIG. 1.

The process flow of FIG. 4 includes an operation 402 of generating a first three-dimensional (3D) model of an internal workspace of a body in which a first portion of an instrument is inserted during performance of a procedure in the internal workspace using the instrument. The instrument may be the same as or similar to the instrument 112 and/or the endoscope 114 illustrated in FIG. 1. Thus, the first portion of the instrument may correspond to a distal end portion of the instrument, which may include an end effector, a wrist, and a first portion of a shaft of the instrument located in the internal workspace 124. The first 3D model may be generated based on image data (e.g., a 3D image data) of the internal workspace 124 corresponding to the reference frame 118, received by the control system 308. The image data of the internal workspace may originate from a first imaging system that is positioned within the internal workspace, such as from the image sensor 116 of the endoscope 114.

The process flow further includes an operation 404 of generating a second 3D model of an external workspace in which a second portion of the instrument is located during the performance of the procedure. The second portion of the instrument may correspond to a proximal portion of the instrument 112 and/or the endoscope 114 that extends from the body wall 110 into the external workspace 122, which may include a proximal portion of the shaft and a transmission mechanism, which in various embodiments can be manually actuatable or configured to operably couple to the manipulator arms 212 in a computer-assisted medical system 200. In some embodiments, the instrument 112 and/or endoscope 114 may be inserted through a cannula, as those having ordinary skill in the art are familiar with, but for simplification purposes, the cannula is omitted in the illustration of FIG. land in the following description.

The second 3D model may be generated based on image data (e.g., 3D image data) of the external workspace 122, received by the control system 308. Operation 404 may include identifying an external boundary locus based on the image data of the external workspace 122, which may correspond to an external portion of the body wall 110. The image data of the external workspace 122 may originate from a second imaging system (e.g., image sensors 102 and/or 220) that is positioned in a room (e.g., mounted to a wall or a ceiling of the operating room, mounted on a manipulator system, such as manipulator system 202, mounted on an auxiliary system such as auxiliary system 206, or located at any other location that can capture an image of a portion of the body wall 110 in which the one or more instruments are inserted and a sufficiently large region of interest surrounding the body wall 110 outside the patient). Knowledgeable persons will understand that multiple instruments may be used in the procedure, and thus multiple first portions of the multiple instruments may be located in the internal workspace and multiple second portions of the multiple instruments may be located in the external workspace.

The process flow includes an operation 406 of determining an internal geometry within the internal workspace 124, the internal geometry defining a reachable volume within the internal workspace 124 within which the instrument 112 and/or endoscope 114 may be positioned. The reachable volume may refer to the total volume within the internal workspace 124 that is defined to be within reach of any one or more of the inserted instruments without resulting in contact or collision, and which in some cases may be based on one or more criteria. The internal geometry may be determined at least based on the first and second 3D models. In some implementations, the internal geometry may be further determined based on a type of medical procedure being performed. In some implementations, the internal geometry may be further determined based on a stage of the medical procedure (e.g., where different stages use different instruments which may have different sizes, working volumes, reachability needs, and other factor that may impact the determination of the internal geometry). The internal geometry may include (or be defined at least in part by) an internal reach boundary that is determined by a locus of positions the end effector of the instrument 112 can reach within the internal workspace 124 but at which an contact between objects in the external workspace 122 would occur, which can include any defined contact as further explained below. In other words, the locus of positions defining an internal reach boundary (with the reachable volume being a volume encompassed from the location of insertion of the instrument and a surrounding portion of the body wall 110 to the internal reach boundary) may correspond to be locations at which, if the portion of the instrument and/or manipulator arm holding an instrument in the external workspace 122 were oriented or moved in such a manner as required to allow the end effector of the instrument 112 to reach the positions, a defined contact in the external workspace 122 would occur. A defined contact may include, without limitation, contact between any two or more manipulator arms (e.g. manipulator arms 212) in the context of a computer-assisted medical procedure utilizing robotic technologies, and/or between a manipulator arm and/or the instrument and an object in the external workspace 122, such as, for example another instrument, another manipulator arm (if any), a portion of the patient's body, a portion of the manipulator system (if any), a table supporting the patient, personnel, other medical equipment supporting the medical procedure, or combinations thereof. Operation 406 may include identifying an internal boundary locus based on, for example, the external boundary locus (identified in operation 404), the internal 3D image data, and the external 3D image data.

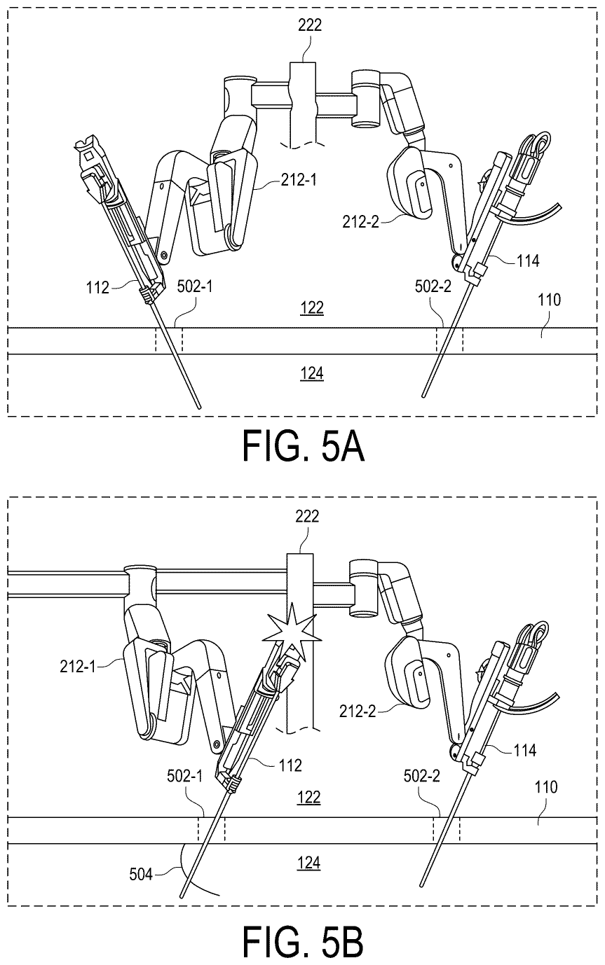

By way of illustration, reference is made to FIGS. 5A and 5B, which show schematically a situation in which the possibility of a defined contact is a criterion informing the determination of the internal reach boundary. In FIGS. 5A and 5B, an illustration is shown with regard to use of the manipulating system 202 of FIG. 2B for carrying out a robotic medical procedure; however, knowledgeable persons will understand that this is merely for purposes of explanation and similar situations may occur if some or all of the instruments or elements shown in FIGS. 5A and 5B were manually-operated. In FIGS. 5A and 5B, as in FIG. 1, a body wall 110 separates the external workspace 122 and the internal workspace 124. A first port 502-1 and a second port 502-2 (collectively “ports 502”) provide access to the internal workspace 124. A medical instrument 112 is mounted to manipulator arm 212-1, which is in turn mounted to the central post 222; and an endoscope 114 is mounted to manipulator arm 212-2, which is in turn also mounted to the central post 222. The instrument 112 and the endoscope 114 extend from the external workspace 122 to the internal workspace 124 via the ports 502. While FIGS. 5A-5B illustrate an example in which manipulator arms 212 are mounted to the central post 222, as noted above one or both of the manipulator arms 212 may be mounted to a wall, ceiling, table, etc.

In FIG. 5A, manipulator arm 212-1 is oriented in a first position such that the distal end portion of instrument 112 is disposed at a particular location within the internal workspace 124. Because there is no contact between the manipulator arms 212, the system may determine that the area where the distal end of instrument 112 is located forms part of the reachable volume. However, in FIG. 5B, manipulator arm 212-1 would need to be oriented in a second position in order for the distal end of the instrument 112 to reach another location within the internal workspace 124. In such a position, however, contact between the manipulator arm 212-1 and the central post 222 would occur. Thus, the system may determine that the area where the distal end of the instrument 112 would be located is not part of (is excluded from) the reachable volume, and may define a portion of an internal boundary locus 504 corresponding to the furthest extent where instrument 112 may be placed (e.g., as determined by a part of the end effector or other distal location of the instrument) without undesirable contact. The internal boundary locus 504 may be located at the actual reach boundary itself, or may be determined by a proximity test (e.g., by adding a predetermined distance as a buffer) so as to incorporate a safety threshold.

Those skilled in the art will understand that, while FIG. 5B illustrates the effect of contact or collision on the reachable internal geometry, in practical situations the reach of one or more instruments 112 may be limited due to kinematic singularities, range of motion limits in the manipulator arms 212, and other factors. For example, if a manipulator arm 212 approaches a singularity, motor speeds can increase and some parts of the manipulator may move in ways that the bedside staff may not anticipate. In this scenario, fast-moving portions of the manipulator arm 212 may present a greater likelihood of contact with an object in the external workspace.

Operation 408 may include providing an output related to performance of the medical procedure based on the determined internal geometry. For example, operation 408 may include outputting information relating to the defined internal boundary locus and/or displaying the internal geometry of the reachable volume to an operator of the instrument (e.g., a surgeon at the user control system 204). The output may include feedback, such as visual feedback (e.g., causing the system 300 to display the internal geometry, a representation of the portion of the instrument within the internal workspace 124, a representation of a contact or potential contact within the external workspace 122, or combinations thereof, to an operator of the instrument), haptic feedback, audio feedback, or combinations thereof. The feedback may include features to distinguish between various types of situations; for example, visual feedback may include different indicators to distinguish different types of contact and/or different levels of risk. By way of non-limiting example, indicators may include visual feedback, for example using colors such as red for patient contact, yellow for robot self-collision, etc.; audio feedback, such as different tones or verbal identifiers to distinguish different types of contact and/or different levels of risk; and/or haptic feedback with differing sensations being associated with different types of contact and/or levels of risk. In some implementations, the type or types of feedback may be selectable by a user.

The process flow of FIG. 4 may be repeated at predetermined intervals and/or on demand, with the system being set up to be used in an automated mode, a manual mode, or a mode allowing to toggle between the two. For example, a manual user request may be made to the control system in a manual mode to recapture internal and/or external 3D image data after a change in the external workspace has been made in response to a reachable volume being identified that does not include a location it is desired to place the instrument in the internal workspace (e.g., undocking one or more manipulator arms that are in a potential defined contact region). Images are then recaptured and the internal geometry model may then be updated, for example by performing some or all of operations 402, 404, 406, and 408 using the new image data. The reachable volume may thereafter be determined and output regarding the same provided so that various planning and actions of the medical procedure can occur.

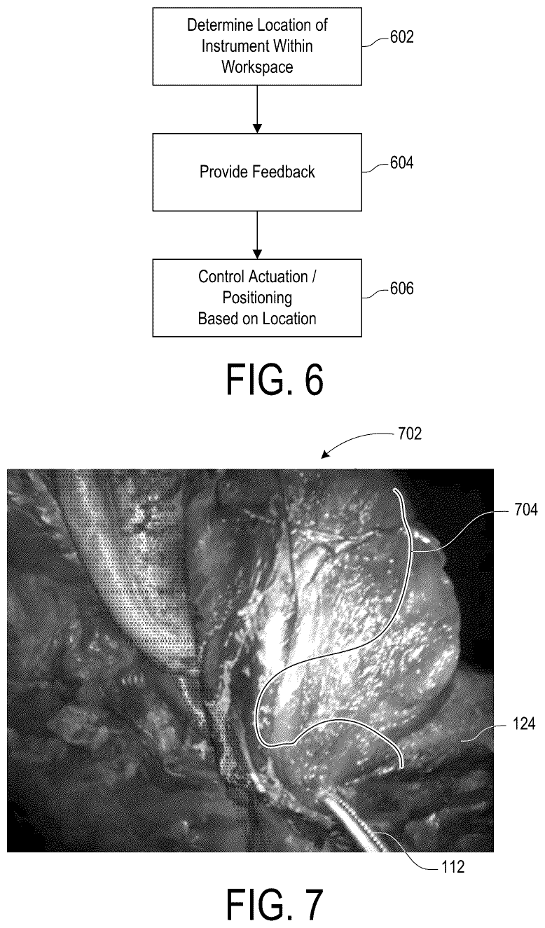

In some implementations, the output provided, for example, at operation 408 in FIG. 4, can include various forms of output (such as feedback and/or control of an instrument, manipulator arm, or other part of a medical system) useful for personnel, such as a surgeon, performing the medical procedure and/or output to control or assist in controlling one or more instruments and/or arms. FIG. 6 illustrates an exemplary process flow which may be based on the reachable workspace geometry and to provide such feedback and/or control. The process flow of FIG. 6 may be performed by, for example, the control system 308. In some implementations, the process flow of FIG. 6 may be embodied in the form of instructions stored in a non-transitory computer-readable medium (e.g., the memory 314) such that the control system 308 performs the operations of FIG. 6 upon execution of the instructions. In other implementations, the process flow of FIG. 6 may be embodied in the form of carrier signals (e.g., provided to the processor 312) such that the control system 308 performs the operations of FIG. 6 upon receipt of the instructions.

The process flow of FIG. 6 may take, as input: orientation, positioning, or other related information received from sensors disposed on one or more of the arms 212, the instruments 112, and/or the endoscope 114; orientation, positioning, or other related information received from sensors associated with the system 300; the internal geometry within the internal workspace 124 (e.g., as generated in operation 406); information regarding system capabilities (e.g., the presence of haptic feedback devices, etc.); information regarding system modes (e.g., whether the system is operating in a manual override mode); information regarding system settings (e.g., user-defined tolerances); the first generated 3D model; the second generated 3D model;

information regarding instrument type; information regarding procedure type; and combinations thereof. The process flow of FIG. 6 includes an operation 602 of determining a location of an instrument or instruments (e.g., 112 and/or 114) within the internal workspace 124 (for example, within the internal geometry) and/or within the external workspace 122.

The process flow of FIG. 6 further includes an operation 604 of providing feedback to an operator of the system (e.g., the surgeon, an assistant, etc.). The feedback may include haptic feedback, for example via haptic feedback devices (e.g., devices 244 of FIG. 2B) associated with a force transmission mechanism of an instrument and/or of a input device at a user control system 204 used by a surgeon to control movement of an instrument. The feedback may include audio feedback, for example via one or more speakers, such as, for example, at the manipulating system 202, the user control system 204, and/or the auxiliary system 206, or anywhere in an audible range of personnel for which such feedback may be useful during a medical procedure. The feedback may additionally or alternatively include visual feedback, for example via the stereoscopic image display system 240, the monitor 214 of FIG. 2B, or other display devices. The visual feedback may take the form of an icon displayed as part of a GUI, a warning or informational message, a request (e.g., a repositioning request to reposition a manipulator arm 212), a light, and the like.

In some implementations, the visual feedback may include displaying an image of a portion of the instrument within the internal workspace, either alone or concurrently with a visualization of the determined internal geometry within the internal workspace. In some implementations, the visual feedback may include displaying of visual indication of a portion (or all) of a boundary of the determined internal geometry. Such representations may include graphical elements corresponding to a boundary that is overlayed with a real-time image corresponding to the output of the endoscope 114 and/or a visualization (e.g., an image or rendering) of the external workspace 122. For example, when distal tip of an instrument end effector or other distal end of an instrument is within a defined proximity of a determined internal reach boundary, the visual feedback may visually highlight the boundary (e.g., by using a graphical contour overlaid on the stereoscopic image captured by the endoscope image device and displayed at the one or more displays of the user control system 204 or auxiliary system 206. FIG. 7 shows one example of visual feedback, in which the control system 308 is configured to display a live video 702 (which includes an image of the internal workspace 124 in which an instrument 112 is located) with a graphical contour 704 overlaid thereon showing the boundary of the determined internal geometry. Operation 604 may be omitted depending on certain system settings. For example, a user may temporarily or persistently disable warning graphics, boundary surface overlays, and so on.

The process flow of FIG. 6 optionally includes an operation 606 of controlling actuation (or positioning) of or assisting in controlling an instrument or arm based on the location determined in operation 602. Operation 606 may include controlling one or more instruments 112 and/or manipulator arms 212 so as to modify (e.g., prevent or slow) certain movement and/or actuation (e.g., actuation of an end effector of an instrument) in at least one direction even if the control system 308 receives a command to perform such movement and/or actuation. For example, even if the control system receives a command from an input control 242 at a user control system 204 and/or other input device (such as at a manually-actuated force transmission mechanism of an instrument) that is operably coupled to control a movement and/or actuation of an instrument and/or portion of the manipulator system 202 (e.g., a foot pedal or other input device configured to provide a command to deliver energy or other type of flux from an instrument), the control system 308 may override the command, for example if it is determined that the instrument is near, at, or past a boundary of the internal workspace 124. Alternatively, operation 606 may include providing a modified control to provide an actuation different from that specified by input at the user control system 204 or a manually-actuated force transmission mechanism of the instrument (not shown) (e.g., actuation, whether of end effector function and/or instrument movement, at a lower speed than commanded or with a delay and feedback to allow the actuation to be backed out of by the user). Operation 606 may be omitted depending on certain system settings. For example, the surgeon may disable actuation control and/or may override the control.

The operations of FIG. 6, which may generally be classified as modifications to permit desired reach, may take one of several different implementations. In one implementation, the control system 308 may provide a visual feedback prompt or guidance to reposition one or more of the instruments 112 and/or manipulator arms 212 (e.g., by displaying a repositioning prompt at a display or via an indicator associated with the arm corresponding to the prompt). Following such a repositioning prompt, repositioning of the relevant instrument(s) and/or arm(s) may occur via an automatic operation through the control system 308 automatically moving the arm 212 into a desired position or via a manual one in which medical personnel physically repositions the arm 212, for example to increase a size of the reachable internal geometry. In the event that the repositioning is automatic, the control system 308 may wait for receipt of a confirmation to carry out the repositioning operation and/or some other responsive input (e.g., pressing a button, actuating a switch (e.g., a dead-man switch), or the like). If multiple instruments 112 and/or arms 212 are to be repositioned, the arms 212 may move one at a time. If, in such a situation, one instrument 112 and/or arm 212 obstructs the repositioning path of another instrument 112 and/or arm 212, the obstructing instrument 112 and/or arm 212 may move first. In the event that the final desired repositioning state is unattainable (e.g., due to proximity of obstacles or the patient), the repositioning operation can be terminated.

In another implementation, the control system 308 may provide a visual prompt, e.g., to one or more members of a medical team, to reposition one or more of the arms 212 in a semi-manual manner, for example via any of the display devices illustrated in FIG. 2B. The control system 308 may illuminate one or more light emitting elements at the appropriate touch points of the arm 212 which is to be moved first. The assistant or other personnel then moves the arm 212 manually. When the arm 212 nears the final repositioning state, the control system 308 may automatically position the arm 212 (e.g., by applying arm brakes) and provide a confirmation notification, such as an audible tone. As above, in the event that the final repositioning state is unreachable (e.g., due to proximity of obstacles or the patient), the assistant or other personnel may terminate the repositioning operation. If additional arms 212 are to be repositioned, the sequence may be repeated. These implementations are presented merely by way of example, and other implementations may instead by utilized.

As part of operation 606, the control system 308 may also be configured to implement a manual boundary correction mode. In such a mode, if a user determines that a predicted internal reach boundary is not correct (e.g., the output of operation 408 of the process flow of FIG. 4 is incorrect or needs recalibration), the system may allow a portion of the instrument 112 or manipulator to be moved, e.g., via a surgeon or bedside staff member, toward the desired location until an actual external boundary is reached or a minimum acceptable clearance is reached. The control system 308 may output a prompt to move any instrument 112 or arm 212 predicted to have a defined contact (e.g., with the patient, with another instrument 112 or arm 212, etc.) toward the patient until the actual safety threshold distance is reached (e.g., a predetermined distance between a portion of the manipulator system 202 and the patient). Once the actual safety threshold distance is reached, the control system 308 may recalculate the body model (e.g., by performing operation 406) within the neighborhood of the new (manually-taught) boundary and then may carry out the remaining operations of FIG. 6.

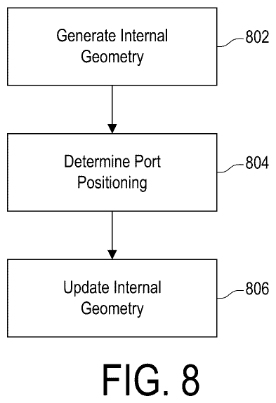

In various other embodiments, the operations of FIG. 4 may additionally or alternatively be used to assist a port planning procedure. The process flow of FIG. 8 illustrates an exemplary port planning process flow in accordance with the present disclosure, which may begin after an initial port has been placed, such as a port for an endoscope. The initial port placement may be based on parameters including but not limited to patient characteristics (e.g., body-mass index or BMI), the type of procedure being performed, the dimensions of an instrument being used for the procedure, previously-acquired images such as CT/MRI images if available, locations of expected ports for manual laparoscopic instruments, and/or a various other factors familiar to those having ordinary skill in the art. FIG. 8 illustrates an operation 802 of generating an initial internal geometry as described above for example with reference to FIG. 4. On an initial pass through the process flow of FIG. 8, operation 802 may include operations 402-408 of FIG. 4. Thus, operation 802 may include sub-operations of generating a first 3D model of an internal workspace 124 of the body in which a first portion of an instrument 112 is to be inserted during performance of a procedure using the instrument and generating a second 3D model of an external workspace 122 in which a second portion of an instrument 112 is located during the performance of the procedure. Operation 802 may also include sub-operations of receiving an imaging data of the internal workspace 124 from an image sensor (e.g., the endoscope 114) and/or receiving an imaging data of the external workspace 122 from another image sensor (e.g., image sensor 102) if the imaging data is unavailable or if it would otherwise be preferable to update the imaging data. For example, the surgeon or other personnel may insert the endoscope 114 into the body via the initial port and sweep the internal workspace 124 to collect internal imaging data. As a result of the initial performance of the operation, operation 802 will generate an initial internal geometry; that is, an internal geometry corresponding to the presence of only the initial port.

The initial internal geometry may be used at operation 804 to determine a port positioning for one or more additional ports (i.e., in addition to the initial port). Operation 804 may be based on a comparison between the initial internal geometry and a target internal geometry (i.e., an internal geometry which includes locations within the internal workspace 124 that are desirable to reach to perform the medical procedure), on a type of procedure being performed, and/or on environmental (external) parameters, for example to ensure that the reachable volume includes all desired locations in the internal workspace. Operation 804 may also be based on the external image data, for example to register the external port placement location with the internal workspace. In general, operation 804 may be based on a volume defined by the internal boundary locus, a probability of defined contact between the instrument and a portion of the body within the internal boundary locus, a probability of defined contact between the instrument 112 and/or associated manipulator arm 212 and an object in the external workspace 122, or combinations thereof. Operation 804 may include providing port planning information. This information may include the planned port positioning itself and/or information relating to possible configurations of the arms 212 and/or instruments 112 in the internal or external workspaces based on the planned port positioning (e.g., a cone of possible or permissible angles of the instruments 112 relative to the corresponding ports). In one example, the information takes the form of a port planning map, which includes one or more locations to place ports configured for insertion of one or more instruments 112 to extend from the external workspace 122 to the internal workspace 124. The port planning map may be displayed to an operator on a display and/or projected directly onto the patient's body using a projector.

Operation 806 includes updating the internal geometry based on the port placement guidance generated in operation 804. Operation 806 may occur immediately after operation 804, or alternatively may occur after one or more additional incisions are made and one or more corresponding additional ports are placed based on the guidance generated in operation 804. In order to update the internal geometry, existing internal image data (e.g., image data of the internal workspace 124 obtained via the endoscope 114 using the initial port) may be used; however, in some implementations additional external and/or internal image data (e.g., image data of the internal workspace 124 obtained via the endoscope 114 using a newly-placed port) may additionally or alternatively be used. Updated image data may be obtained at a predetermined interval, on demand, or in real-time. Operations 804 and 806 may be repeated in an iterative or consecutive manner until a final port placement map is produced, which includes positioning information for a sufficient number of ports such that a desired reachable volume by one or more instruments 112 is achieved. In such implementations, each iteration of operation 804 may generate a planned position for one (or, in some examples more than one) additional port based on updated internal geometry information from a previous iteration of operation 806. The updated port placement map may then be used in a subsequent iteration of operation 806 to generate newly-updated internal geometry information, which may then be used in a subsequent operation 804, and so on until the final port placement map is achieved. In an example, these iterative operations may be performed in or near real-time. For example, a user may move a pointer (e.g., a finger, obturator, or other device) around the body wall 110 near planned port locations, and operation 806 may be continuously performed to update the internal reach boundary in real-time.

While FIGS. 4, 6, and 8 illustrate operations in a particular order, in practical implementations the operations may be performed in any order so long as an operation which takes an input occurs after an operation which generates the input (e.g., operations 402 and 404 are performed prior to operation 406). Additionally, some of the operations may be performed continually and/or continuously, for example by continually performing operations 402-408 to update the 3D model of the internal workspace 124 and/or the 3D model of the external workspace 122, and to continuously (dynamically) adjust the reachable volume of a given instrument during a medical procedure, and to provide an output based on the reachable volume continuously The dynamic adjustment may be performed in response to one or more triggering events, including but not limited to an instrument change, a change in environment, a change in configuration (e.g., a change in pose) of the manipulator system or instrument, a change in operating table orientation and/or configuration, introduction of additional medical equipment such as a mobile X-ray machine, moving of tables, a change in a stage of the medical procedure (which may itself include or be associated with one or more of the changes listed here), and the like. Moreover, in some implementations one or more of the operations may be performed in parallel. For example, in FIG. 4 the control system 308 may simultaneously generate the 3D model of the internal workspace 124 and the 3D model of the external workspace 122 using the same or different processing resources. In some implementations, one or more of the operations may be performed multiple times. For example, in FIG. 4 an initial iteration of operation 402 may be performed to generate an initial 3D model of the internal workspace 124, and subsequent iterations of operation 402 may be performed to update the initial 3D model of the internal workspace 124 and/or to generate a new, updated 3D model of the internal workspace 124.

One or more of the operations illustrated in FIG. 4, 6, or 8 may be performed manually (e.g., by a surgeon, an assistant, and/or other personnel), automatically or computer-assisted (e.g., by the manipulating system 202, the user control system 204, the auxiliary system 206, components thereof, and/or other equipment), or a combination thereof. In one particular example, the operations of FIGS. 4 and 8 may be performed in a combined manual and computer-assisted manner as follows. In the example, operation 404 may be performed first to generate a 3D model of the external workspace using a depth sensing camera mounted on an overhead lamp (an example of the external image sensor 102 of FIG. 1 and/or the external image sensor 220 of FIG. 2B). The depth sensing camera may scan the patient and a reference point set with a marker (e.g., the patient's navel). Based on the 3D model of the external workspace, the system performing the operations may provide an indication of a proposed first entry point (i.e., an initial port placement), which may be based on factors including but not limited to the procedure being performed, the stage of the procedure, or patient data. Medical personnel performing the procedure, such as the surgeon or assistant, may manually palpate the anatomy to verify the proposed initial port placement. These operations may be guided by a live video based on image data from the depth sensing camera, in an example of the partial performance of operation 408. If the medical personnel confirms that the proposed initial port placement is correct or viable, the medical personnel (or another medical personnel) may make an incision at the first entry point and insert an imaging device, such as a trocar and laparoscope (an example of the internal image sensor 116 of FIG. 1).

By manually operating the laparoscope, the medical personnel may perform operation 402 to scan the interior of the patient and generate a 3D model of the internal workspace. The medical personnel may manipulate the laparoscope so as to obtain image data for one or more desired features of the internal workspace, such as a target of the procedure, organs of the patient, or bony areas. These operations may be guided by a live video based on image data from the laparoscope, in an example of the partial performance of operation 408. Based on image data of the external and internal workspaces, operation 406 may be performed. In this example, the depth sensing camera may track the laparoscope, for example by detecting an insignia (e.g., a QR code) on a handle of the laparoscope. Thus, operation 406 may include registration between the external workspace and the internal workspace using the systems and methods discussed in International Patent Application No. PCT/US2021/065444, filed on Dec. 29, 2021, now published as WO 2022/147074, incorporated by reference above. These operations may thus correspond to sub-operations of operation 802 as described above.

Subsequently in this example, the medical personnel may view the internal geometry and select a target workspace. In one particular example, the medical personnel may draw a target boundary on a touchpad associated with the system, thereby to define the target workspace. Thereafter, the system may proceed to determine the port position (an example of operation 804), such as by automatically testing all possible port placements for compatibility with the external workspace (e.g., to avoid undesired or unintended contact) and/or the internal workspace (e.g., to achieve the target workspace with a minimum number of incisions). The system may generate a port placement map and may display the same for the medical personnel. The display may be performed on a display device, such as the touchpad used by the medical personnel to select the target workspace, or by projecting the map onto the patient. The medical personnel may then mark and/or create the additional incisions while viewing a live image of the external workspace using the depth sensing camera.

One or more of the operations illustrated in FIG. 4, 6, or 8 may include a sub-operation of tracking an object (e.g., an instrument, a manipulator arm, a body portion, and/or another internal or external object) using images exterior and interior to the body. The control system 308 may receive one or more images from one or more external image sensors 102/220 and one or more images from the internal image sensor 116. The control system 308 may perform a registration to determine a correspondence (e.g., including alignment relationships of partial or full degrees of freedom) between images provided by these image sensors, and transform the image data to a common reference frame. In some examples, such a registration may involve determining the three degrees of freedom translation and three degrees of freedom transformation (or a subset of these degrees of freedom) between the image sensors' field(s) of view and the common reference frame. Using the transformed image data from external image sensors 102/220 and/or internal image sensor 116, the control system 308 may track the object's movement and generate a tracking result to indicate a status of movement of the object. In various implementations, the tracking result may indicate the object being tracked, a moving direction of the object, a total number of sub-portions of the object that have been moved, a location of the object relative to another object, and the like.

In examples, the control system 308 may determine a tracking configuration based on the object to be tracked and the operation anticipated or in process, and perform the tracking using the tracking configuration. For example, the control system 308 may determine a tracking configuration based on a type of the object (e.g., an instrument, a manipulator arm, a body portion, and/or another internal or external object). As another example, the operation tracked may include the object entering or exiting the body (e.g., an instrument entering and/or exiting the body; removing and/or transplanting a body portion; removing, placing, and/or replacing an implant, etc.).

In the tracking operation, the control system 308 may receive sensor data including external image data from the one or more external image sensors 102/220 and/or including internal image data from the internal image sensor 116. The external or internal image data may include an object to track (e.g., an instrument, an instrument tip, a manipulator arm, a portion of the body, a room feature, etc.) or a representation/model of the object to track. The object to track may be identified in the internal image data by markings, colors, shapes, sizes/dimensions, any other suitable features, associations with equipment that may be interacting with the object, attributes or features identified by a machine learning model, and/or a combination thereof. In some embodiments, the control system 308 may use the same feature(s) for identifying the object to track in the external and internal image data. Alternatively, in some embodiments, the control system 308 may use different feature(s) for identifying the object to track in the external and internal image data, for example based on the different image properties (e.g., imaging conditions, image resolution, image metadata, etc.) of the first and second image data. In some embodiments, the views for an external image sensor 102/220 and an internal image sensor 116 may be mutually exclusive (e.g., one sees inside the body while the other sees outside the body), and the tracked object may not be included in both first image data and second image data captured at the same time. In such embodiments, a synthetic (e.g., based on a model of the object to track) overlay of the object to track (e.g., based on information from one image data) may be provided in the other image data where the object to track is not directly visible.

The tracking operation may further include determining a registration between the external image data and the internal image data. In such implementations, the control system 308 may determine a registration between the external image sensor(s) 102/220 and the internal image sensor 116. In some embodiments, the registration may be performed by registering the image sensors to manipulator arms coupled to the image sensors (e.g., where an external image sensor 102/220 is mounted on a manipulator arm 212 or where the internal image sensor 116 is manipulated by a manipulator arm 212), and registering the manipulator arms to each other. Various image registration methods may also be used by the control system to determine such an alignment relationship using the external and internal image data. In some embodiments, the registration is performed further using additional image data (e.g., pre-and intra-operative image data, computed patient mesh, etc.), and the registered set of images includes external and internal image data from the external and internal image sensors and those registered additional image data. The control system 308 may transform the external and/or internal image data according to the alignment relationship to a common reference frame. In some embodiments, the common reference frame may be a 3D coordinate system coincident with the reference frame of either imaging sensor, the 3D coordinate system of the manipulator assembly, or 2D image planes of either image sensor.

The control system 308 may further track the object relative to one or more other objects (e.g., an instrument, an instrument tip, a manipulator arm, a portion of the body, a room feature, etc.) based on the external and/or internal image data and the registration to generate a tracking result to indicate whether there is a likelihood of undesired contact or near-contact between the tracked object and the one or more other objects. In an example, the control system 308 may generate one or more movement paths of the tracked object and/or other object(s). The movement paths may be represented using multiple images or video images. In some embodiments, when the object to track is not present directly in either the external or internal image data, or when the object to track is present but occluded in either the external or internal image data, an estimate of its position and motion may be generated based on its past recorded positions and/or motions in either image data.

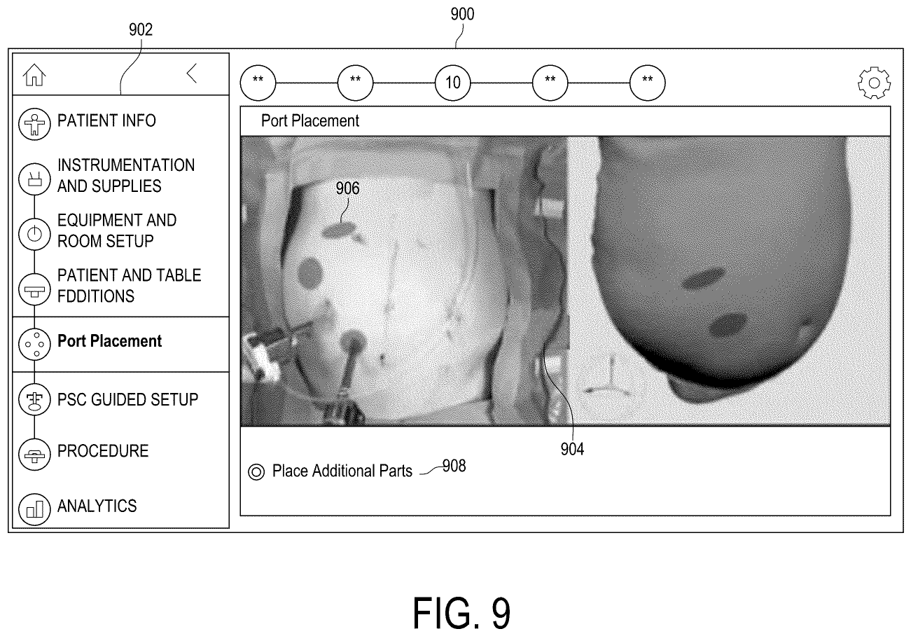

FIG. 9 illustrates an exemplary GUI 900 which may be presented during performance of a medical procedure and/or a medical procedure preparation. In some examples, the GUI 900 presents the information produced by one or more of the process flows of FIGS. 4, 6, and/or 8 to the operator. The GUI 900 may be displayed using the display 240 and/or the monitor 214 of FIG. 2B, or any other display screens to be used during a medical procedure, or combinations thereof. The GUI 900 includes a menu section 902 and a video display section 904. The menu section 902 may present a list of menu items to the operator, thereby to permit the operator to switch between various views and screens. Based on the selected menu item, the video display section 904 displays the appropriate image data. For example, the menu section 902 may permit a selection between a patient information screen in which patient information may be entered and/or displayed, an instrumentation screen in which information relating to the types of instruments 112 used for the medical procedure may be entered and/or displayed, an equipment and room screen in which information relating to the types of equipment used and/or the operating room may be entered and/or displayed, an orientation screen in which information relating to the position and orientation of the patient, operating table, etc. may be entered and/or displayed, a port placement screen in which information relating to the position and/or planned position of surgical ports may be entered and/or displayed a procedure screen in which information relating to the procedure may be entered and/or displayed, a setup screen in which information relating to various settings, operations, and/or preferences may be entered and/or displayed, and an analytics screen in which various performance metrics may be entered and/or displayed. Individual portions of the GUI 900 may be used independently of the other portions of the GUI 900. For example, an operator may select which screen to display using the menu section 902, which may cause the GUI 900 to update only the video display section 904 while retaining other portions of the GUI 900.

In the illustration of FIG. 9, the port placement screen has been selected, such that port placement information is presented on video display section 904. In this example, the video display section 904 presents an actual video feed of the portion of a patient body wall in the external workspace that is accessible for port placement to perform the medical procedure (left side of section 904) and a 3D rendering of the portion of the patient body wall (right side of section 904). A series of port locations 906 are overlaid on both the actual video feed and the 3D rendering, for example, as an overlay, which in some cases may be partially transparent so as to not obstruct the video image of the patient body. Some or all of the port locations 906 may be associated with an identifier (e.g., a different color, shape, hatching, etc.) if desired, for example to identify an endoscope port. The video display section 904 also includes an input field 908 which the operator may use to provide input, for example to initiate various operations, including but not limited those described above with regard to FIGS. 4, 6, and 8. In other examples, the video display section 904 may present only a single video and/or may show information regarding the internal workspace.

Various aspects of the present disclosure as described herein may be well suited for use in any of a variety of medical procedures for which it may be desirable and/or advantageous to obtain and/or use a reachable volume of an internal workspace, as described above. Such procedures could be performed, for example, on human patients, animal patients, human cadavers, animal cadavers, and portions or human or animal anatomy. Medical procedures as contemplated herein include any of those described herein and include, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the aspects may also be used for benchtop procedures on non-living material and forms that are not part of a human or animal anatomy. Moreover, some aspects are also suitable for use in non-medical applications, such as industrial robotic uses, and sensing, inspecting, and/or manipulating non-tissue work pieces. In non-limiting aspects, the techniques, methods, and devices described herein may be used in, or may be part of, a computer-assisted surgical system employing robotic technology such as various da Vinci® Surgical Systems and Ion Endoluminal System commercialized by Intuitive Surgical, Inc., of Sunnyvale, California. Those skilled in the art will understand, however, that aspects disclosed herein may be embodied and implemented in various ways and systems, including manually operated minimally invasive medical systems and computer-assisted, teleoperated systems, in both medical and non-medical applications. Reference to the da Vinci® Surgical Systems are illustrative and not to be considered as limiting the scope of the disclosure herein.

As used herein and in the claims, terms such as computer-assisted manipulating system, manipulating system, or variations thereof should be understood to refer broadly to any system comprising one or more controllable kinematic structures (“manipulators”) comprising one or more links coupled together by one or more joints that can be operated to cause the kinematic structure to move. Such systems may occasionally be referred to in the art and in common usage as robotically assisted systems or robotic systems. The manipulators may have an instrument permanently or removably mounted thereto and may move and operate the instrument. The joints may be driven by drive elements, which may utilize any convenient form of motive power, such as but not limited to electric motors, hydraulic actuators, servomotors, etc. The operation of the manipulator may be controlled by a user (for example through teleoperation), by a computer automatically (so-called autonomous control), or by some combination of these. In examples in which a user controls at least some of the operations of the manipulator, an electronic controller (e.g., a computer) may facilitate or assist in the operation. For example, the electronic controller may “assist” a user-controlled operation by converting control inputs received from the user into electrical signals that actuate drive elements to operate the manipulators, providing feedback to the user, enforcing safety limits, and so on. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems may be implemented in a variety of contexts to perform a variety of procedures, both medical and non-medical. Thus, although some examples described in greater detail herein may be focused on a medical context, the devices and principles described herein are also applicable to other contexts, such as industrial manipulator systems.

It is to be understood that both the general description and the detailed description provide example aspects that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the aspects.

Further, the terminology used herein to describe aspects of the present disclosure, such as spatial and relational terms, is chosen to aid the reader in understanding various aspects of the present disclosure but is not intended to limit the present disclosure. For example, spatial terms-such as “beneath,” “below,” “lower,” “above,” “upper,” “proximal,” “distal,” “up,” “down,” and the like-may be used herein to describe directions or one element's or feature's spatial relationship to another element or feature as illustrated in the drawings. These spatial terms are used relative to the drawings and are not limited to a particular reference frame in the real world. Thus, for example, the direction “up” in the drawings does not necessarily have to correspond to an “up” in a world reference frame (e.g., away from the Earth's surface). Furthermore, if a different reference frame is considered than the one illustrated in the drawings, then the spatial terms used herein may need to be interpreted differently in that different reference frame. For example, the direction referred to as “up” in relation to one of the drawings may correspond to a direction that is called “down” in relation to a different reference frame that is rotated 180 degrees from the drawing's reference frame. As another example, if a device is turned over 180 degrees in a world reference frame as compared to how it was illustrated in the drawings, then an item described herein as being “above” or “over” a second item in relation to the drawings would be “below” or “beneath” the second item in relation to the world reference frame. Thus, the same spatial relationship or direction can be described using different spatial terms depending on which reference frame is being considered. Moreover, the poses of items illustrated in the drawings are chosen for convenience of illustration and description, but in an implementation in practice the items may be posed differently. In general, “distal” as used herein refers to a direction toward an end effector of an instrument or other free working end of a kinematic chain, and “proximal” refers to the opposite direction.

In addition, the singular forms “a,” “an,” and “the” are intended to include the plural forms as well, unless the context indicates otherwise. Additionally, the terms “comprises,” “comprising,” “includes,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one aspect may, whenever practical, be included in other aspect in which they are not specifically shown or described. For example, if an element is described in detail with reference to one aspect and is not described with reference to a second aspect, the element may nevertheless be claimed as included in the second aspect.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances. Furthermore, the range of variation also includes at least variations that are within ±5% of the stated value, property, or relationship. Thus, for example, a line or surface may be considered as being “approximately parallel” to a reference line or surface if any one of the following is true: the smallest angle between the line/surface and the reference is less than or equal to 4.5° (i.e., 5% of 90°), the angle is less than or equal to manufacturing or other tolerances typical in the art, or the line/surface as constituted is functionally equivalent to the line/surface if it had been perfectly parallel.

Further modifications and alternative aspects will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various aspects shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and aspects set forth herein are non-limiting, and modifications to structure, dimensions, materials, and methodologies may be made without departing from the scope of the present teachings.

Other aspects in accordance with the present disclosure will be apparent to those skilled in the art from consideration of the specification and practice of the present disclosure disclosed herein. It is intended that the specification and examples be considered as exemplary only, with the following claims being entitled to their fullest breadth, including equivalents, under the applicable law.

What is claimed is:

1. A computer-assisted system, comprising:
an instrument configured to be at least partially inserted through a body wall from an external workspace to an internal workspace within a body; and
a controller configured to:
generate a first three-dimensional model of the internal workspace in which a first portion of the instrument is inserted during performance of a medical procedure using the instrument,
generate a second three-dimensional model of an external workspace in which a second portion of the instrument is located during the performance of the medical procedure,
based on the first three-dimensional model and the second three-dimensional model, determine an internal geometry within the internal workspace defining a reachable volume within the internal workspace within which the instrument may be positioned, and
provide output related to performance of the medical procedure based on the determined internal geometry.

2. The system of claim 1, further comprising a manipulator arm operatively coupled to the instrument and located in the external workspace, wherein the controller is further configured to control drive force transmission from the manipulator arm to the instrument.

3. The system of claim 2, wherein the internal geometry includes an internal reach boundary, the internal reach boundary being a boundary defining a location of the first portion of the instrument within the internal workspace at which one or both of the instrument or manipulator arm contacts another object within the external workspace.

4. The system of claim 2, wherein the controller is further configured to:
determine a location of an instrument within the internal workspace; and
display a repositioning request, the repositioning request including a visual indication to reposition the manipulator arm.

5. The system of claim 2, wherein the controller is further configured to generate guidance for adjusting the manipulator arm to increase a size of the internal geometry.

6. The system of claim 1, wherein the internal geometry is further determined based on one or both of a type of the medical procedure using the instrument or the stage of the medical procedure.

7. The system of claim 1, wherein the controller is further configured to cause the system to:
determine a location of the instrument within the internal workspace, and provide a feedback to an operator of the instrument based on the location of the instrument.

8. The system of claim 7, wherein the feedback includes a haptic feedback, a visual feedback, an audio feedback, or a combination thereof.

9. The system of claim 1, wherein the controller is further configured to cause the system to:
determine a location of the instrument within the internal workspace, and
control actuation of the instrument based on the location of the instrument within the internal workspace.

10. The system of claim 1, wherein the controller is further configured to cause the system to:
based on the first three-dimensional model and the second three-dimensional model, identify one or more locations on an external surface of the body for insertion of the instrument to access the internal workspace during the procedure.

11. The system of claim 1, wherein the controller is further configured to:
receive internal three-dimensional image data of the internal workspace,
receive external three-dimensional image data of the external workspace, wherein a boundary is defined between the internal workspace and the external workspace and comprises an external surface portion of the body,
identify an external boundary locus based on the external three-dimensional image data,
define an internal boundary locus based on at least the identified external boundary locus and the internal three-dimensional image data, and
output information relating to the defined internal boundary locus.

12. The system of claim 11, wherein the controller is further configured to cause the system to display the information relating to the defined internal boundary locus, and the information includes a partially-transparent graphical element corresponding to the internal boundary locus that is overlayed with a real-time image.

13. The system of claim 1, wherein the controller is further configured to:
based on the first three-dimensional model and the second three-dimensional model, provide information relating to planning a positioning of the instrument during the procedure, and
update the first three-dimensional model based on an updated output of an image sensor.

14. A computer-implemented method, comprising:
generating a first three-dimensional model of an internal workspace of a body in which a first portion of an instrument operatively coupled to a manipulator arm configured to transmit drive force to the instrument is inserted during performance of a procedure using the instrument;
generating a second three-dimensional model of an external workspace in which a second portion of the instrument is located during the performance of the procedure;
based on the first three-dimensional model and the second three-dimensional model, determining an internal geometry within the internal workspace defining a reachable volume within the internal workspace within which the instrument may be positioned by the manipulator arm; and
providing output related to performance of the procedure based on the determined internal geometry.

15. The method of claim 14, wherein the internal geometry includes an internal reach boundary, the internal reach boundary being a boundary defining a location of the first portion of the instrument within the internal workspace at which one or both of the instrument or manipulator arm contacts another object within the external workspace.

16. The method of claim 14, further comprising displaying the internal geometry, a representation of the first portion of the instrument within the internal workspace, or both to an operator of the instrument.

17. The method of claim 14, further comprising:
determining a location of the instrument within the internal workspace, and
providing a feedback to an operator of the instrument based on the location of the instrument.

18. The method of claim 14, further comprising:
determining a location of the instrument within the internal workspace, and
controlling actuation of the instrument based on the location of the instrument within the internal workspace.

19. The method of claim 14, further comprising:
based on the first three-dimensional model and the second three-dimensional model, identifying one or more locations on an external surface of the body for insertion of the instrument to access the internal workspace during the procedure.

20. The method of claim 14, further comprising:
receiving internal three-dimensional image data of the internal workspace,
receiving external three-dimensional image data of the external workspace, wherein a boundary is defined between the internal workspace and the external workspace and comprises an external surface portion of the body,
identifying an external boundary locus based on the external three-dimensional image data,
defining an internal boundary locus based on at least the identified external boundary locus and the internal three-dimensional image data, and
outputting information relating to the defined internal boundary locus.
